(19) United States
(12) Patent Application Publication
(10) Pub. No.: US 2023/0220391 A1
(43) Pub. Date: Jul. 13, 2023

(54) CILP-1 Inhibitors for Use in the Treatment of Dilated Cardiomyopathies

(71) Applicants: Genethon, Evry-Courcouronnes (FR); Universite d'Evry Val d'Essonne, Evry (FR); INSERM (INSTITUT NATIONAL DE LA SANTÉ ET DE LA RECHERCHE MÉDICALE), Paris (FR)

(72) Inventors: Isabelle Richard, Corbeil-Essonnes (FR); Ariane Biquand, Maisons Alfort (FR)

(73) Assignees: Genethon, Evry-Courcouronnes (FR); Universite d'Evry Val d'Essonne, Evry (FR); Institut National de la Santé et de la Recherche Médicale, Paris (FR)

(21) Appl. No.: 18/009,018

(22) PCT Filed: Jun. 9, 2021
(86) PCT No.: PCT/EP2021/065428

§ 371 Date: Dec. 8, 2022

(30) Foreign Application Priority Data
Jun. 9, 2020 (EP) 20305623.9

(51) Int. Cl. C12N 15/113; A61P 9/04; C12N 15/86

(52) U.S. Cl. CPC C12N 15/113; A61P 9/04; C12N 15/86; C12N 2310/531; C12N 2310/14; C12N 2320/32; C12N 2750/14143; C12N 2750/14171

(57) ABSTRACT

The present disclosure relates to the treatment of dilated cardiomyopathies, in particular to 5 the use of an inhibitor of CILP-1.

### FIELD OF THE INVENTION

The present disclosure relates to the treatment of dilated cardiomyopathies, in particular to the use of an inhibitor of CILP-1.

### BACKGROUND OF THE INVENTION

Cardiomyopathy and heart failure remain, despite management, one of the major causes of morbidity and mortality worldwide. Dilated cardiomyopathies (DCM or CMD) are characterized by hypokinesis of the myocardium and dilatation of the cardiac cavities. The cardiac remodelling that takes place during dilated cardiomyopathies consists of damage to the cardiomyocytes associated with the presence of fibrosis, which are inseparable from each other. The damage to the cardiomyocytes involves a decrease in their contractile capacity and a change in their structure, which leads to apoptosis and to the expansion of fibrosis, which replaces the necrotic cardiomyocytes. The proliferation of fibroblasts prevents compensatory hypertrophy of the cardiomyocytes. These manifestations will clinically translate into a decrease in cardiac function. This serious complication can be a cause of death.

Causes include in particular genetics, and a variety of toxic, metabolic or infectious agents. Coronary artery disease and high blood pressure may play a role, but are not the primary cause. In many cases the cause remains unclear. The exact mechanism of cardiomyocyte involvement depends on the etiology of the disease. In genetically-induced dilated cardiomyopathies, most of the genes involved code for structural elements of cardiomyocytes, including extracellular matrix or Golgi apparatus proteins (laminin, fukutin) involved in cellular adhesion and signaling pathways; desmosome proteins (desmocollin, plakoglobin) involved in cellular junctions; sarcoplasmic reticulum proteins (RYR2, ATP2A2, phospholamban) involved in calcium homeostasis; nuclear envelop proteins (lamin A/C) involved in myocardial structural organisation; cytoskeleton proteins (dystrophin, telethonin, α-actinin, desmin, sarcoglycans) involved in cytoskeleton integrity and muscular strength transmission; and sarcomer proteins (titin, troponin, myosin, actin) involved in generation and transmission of muscular strength. In Duchenne muscular dystrophy (DMD), a muscle disease due to mutation in the dystrophin gene, a dilated cardiomyopathy is clinically revealed around the age of 15 years and affects almost all patients after the age of 20 years. In the case of Becker muscular dystrophy (BMD), an allelic form of DMD, cardiac damage develops at the age of 20 years, and 70% of patients are affected after the age of 35. DMC due to titin, a giant protein of the sarcomere, is implicated in 1/250 cases of heart failure (Burke et al., JCI Insight. 2016; 1(6):e86898).

The drugs currently available for the treatment of dilated cardiomyopathies will improve the symptoms but not treat the cause of the disease. The treatments prescribed are those for heart failure, accompanied by hygienic and dietetic measures such as reducing alcohol consumption, reducing water and salt intake and moderate and regular physical exercise. Among pharmaceutical treatments, angiotensin II converting enzyme inhibitors (ACE inhibitors) prevent the production of angiotensin II in order to decrease vasoconstriction and blood pressure. Diuretic drugs remove excess salt and water from the body by inhibiting renal sodium reabsorption. β-blockers, or β-adrenergic receptor antagonists, block the effects of adrenergic system mediators stimulated during dilated cardiomyopathies and decrease heart rate. Mineral-corticoid receptor antagonists block the binding of aldosterone and lower blood pressure. When rhythm disturbances are severe, anti-arrhythmic drugs such as amiodarone are prescribed. Implantation of a pacemaker and/or automatic defibrillator may also be considered. In the most severe cases, patients may benefit from a heart transplant (Ponikowski, et al. 2016, European Heart Journal, 37, 2129-2200).

These approaches are therefore also valid for the management of dilated cardiomyopathies in cases of DMD and titinopathies. There is currently no curative treatment for these pathologies. Corticosteroid treatment, frequently prescribed in DMD, allows an improvement of the muscular phenotype in the medium term thanks to a reduction in inflammation, but its action on the cardiac phenotype is subject to debate. The management of DMD related to dystrophin and titin requires an annual and systematic cardiac check-up (electrocardiogram and ultrasound). In particular, Perindopril, an angiotensin-converting enzyme inhibitor, has been shown to reduce mortality in DMD patients when taken as a preventive treatment from childhood onwards (Duboc, D., et al. 2005. Journal of the American College of Cardiology 45, 855-857).

Molecules being tested in the treatment of cardiac impairment in DMD are mainly molecules already used in the treatment of heart failure. Other therapies are aimed at treating muscle and heart damage by reducing fibrosis. This is the case for Pamrevlumab (Phase II trial NCT02606136), a monoclonal antibody directed against connective tissue growth factor, and Tamoxifen (Phase I trial NCT02835079 and Phase III trial NCT03354039), an anti-estrogen. There is therefore a medical need to develop new therapeutic strategies for dilated cardiomyopathies.

CILP-1 is a matrix-cellular protein found mainly in the chondrocytes of articular cartilage, but its expression has recently been found to be significantly higher in human idiopathic dilated cardiomyopathy and infarction (Yung, C. K., et al. 2004. Genomics, 83, 281-297; van Nieuwenhoven, et al. 2017. Scientific Reports, 7, 16042).

In the hearts of normal mice, CILP-1 is expressed by cardiomyocytes and fibroblasts, the protein is found in the cytosol, nuclear fraction and extracellular matrix (Zhang, C.-L, et al. 2018. Journal of molecular and cellular cardiology 116, 135-144; van Nieuwenhoven, et al. 2017. Scientific Reports 7, 16042). The expression of the CILP-1 protein is increased in a mouse model of induced cardiac fibrosis and its expression is stimulated by TGF-β1 (Mori, M., et al. 2006. Biochemical and biophysical research communications, 341, 121-127). CILP-1 appears to have a positive effect on cardiac remodelling, notably through its inhibitory effect on TGF-β activity via the SMAD signalling pathway in cells by binding to TGF-β1 (Zhang, C.-L, et al. 2018, Journal of molecular and cellular cardiology, 116, 135-144, van Nieuwenhoven, et al. 2017. Scientific Reports 7, 16042, Shindo, K. et al. 2017, International Journal of Gerontology, 11, 67-74).

### SUMMARY OF THE INVENTION

The inventors have found that CILP-1 expression is overexpressed in two developed models of genetically-induced dilated cardiomyopathy, Duchenne muscular dystrophies (DBA2mdx mice) and titinopathies (DeltaMex5 mice). Using the DeltaMex5 mice model which is a severe model of dilated cardiomyopathies, and contrary to previous studies, the inventors have surprisingly shown that inhibition of CILP-1 expression in DeltaMex5 mice showed significant improvement in cardiac fibrosis and decrease of heart hypertrophy. These results indicated that inhibition of CILP-1 represents a therapeutic approach for dilated cardiomyopathy, in particular genetically-induced cardiomyopathy such as titinopathy for which gene transfer approaches are not possible because of the size of the gene.

The present invention relates to a CILP-1 inhibitor for use in the treatment of dilated cardiomyopathies. In a particular embodiment, the CILP-1 inhibitor is a nucleic acid interfering with CILP-1 expression, preferably a shRNA. Said shRNA can be encoded by a nucleic acid construct, preferably comprising at least one sequence selected from the group consisting of: SEQ ID NO: 1 to 4.

In a preferred embodiment, said nucleic acid construct is packaged into a viral particle, more preferably an adeno-associated viral (AAV) particle. In a particular embodiment, said nucleic acid construct packaged into the AAV particle comprises 5′-ITR and 3′-ITR of AAV-2 serotype. In another particular embodiment, said AAV capsid protein is derived from AAV serotypes selected from the group consisting of: AAV serotypes 1, 6, 8, 9 and AAV9.rh74, preferably AAV-9.rh74 serotype. In a particular embodiment, said viral particle is administrated intravenously.

The dilated cardiomyopathy according to the invention is preferably a genetically induced cardiomyopathy caused by mutation(s) in a gene selected from the group consisting of: laminin, emerin, fukutin, fukutin-related protein, desmocollin, plakoglobin, ryanodine receptor 2, sarcoplasmic reticulum ca(2+) ATPase 2 isoform alpha, phospholamban, lamin a/c, dystrophin, telethonin, actinin, desmin, sarcoglycans, titin, myosin, RNA-binding motif protein 20, BCL2-associated athanogene 3, desmoplakin, sodium channels, cardiac actin, cardiac troponin, and tafazzin, preferably caused by mutation in titin or dystrophin

In another aspect, the present invention relates to a pharmaceutical composition comprising the CILP-1 inhibitor and a pharmaceutical excipient for use in the treatment of dilated cardiomyopathies.

### DETAILED DESCRIPTION OF THE INVENTION

The present disclosure relates to a CILP-1 inhibitor for use in treating a dilated cardiomyopathy in a subject in need thereof.

The gene Cartilage intermediate layer protein (CILP-1) (Gene ID: 8483) encodes for a CILP-1 preprotein (Accession number: NP_003604.4) for two different proteins, CILP-1 (Accession numbers: XP_016878168.1 or XP_016878167.1) and C-terminal homolog of NTPPHase. The gene sequences of a number of different mammalian CILP-1 proteins are known including, but being not limited to, human, pig, chimpanzee, dog, cow, mouse, rabbit or rat, and can be easily found in sequence databases.

By “CILP-1 inhibitor” is meant any agent able to decrease specifically CILP-1 expression and/or biological activity, in particular that results in inhibition of TGFβ.

The CILP-1 expression and/or activity can be decreased by agents including, but are not limited to, chemicals, compounds known to modify gene expression, modified or unmodified polynucleotides (including oligonucleotides), polypeptides, peptides, small RNA molecules and interfering nucleic acid molecule.

CILP-1 inhibitor can be identified by measuring the decrease of CILP-1 activity, in particular by measuring the expression level of TGF-β in a cell treated with said CILP-1 inhibitor. The CILP-1 activity is decreased in cells when the expression level of TGFβ is at least 1.5-fold lower, or 2, 3, 4, 5-fold lower than in non-treated cells.

CILP-1 inhibitor can be identified by measuring the expression level of CILP-1 in a cell. CILP-1 expression level is decreased in a cell treated with said CILP-1 inhibitor when the expression level of CILP-1 is at least 1.5-fold higher, or 2, 3, 4, 5-fold lower than in non-treated cells. The expression level of CILP-1 mRNA or protein may be determined by any suitable methods known by skilled persons as described above.

The expression level of CILP-1 mRNA may be determined by any suitable methods known by skilled persons. For example the nucleic acid contained in the sample is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). The expression level of CILP-1 protein may also be determined by any suitable methods known by skilled persons. The quantity of the protein may be measured, for example, by semi-quantitative Western blots, enzyme-labelled and mediated immunoassays, such as ELISAs, biotin/avidin type assays, radioimmunoassay, immunoelectrophoresis, mass spectrometry or immunoprecipitation or by protein or antibody arrays.

Interfering Nucleic Acid

In a particular embodiment, said CILP-1 inhibitor may be an interfering nucleic acid which specifically decreases CILP-1 expression.

The terms “nucleic acid sequence” and “nucleotide sequence” may be used interchangeably to refer to any molecule composed of or comprising monomeric nucleotides. A nucleic acid may be an oligonucleotide or a polynucleotide. A nucleotide sequence may be a DNA or RNA. A nucleotide sequence may be chemically modified or artificial. Nucleotide sequences include peptide nucleic acids (PNA), morpholines and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3P5′-phosphoramidates and oligoribonucleotide phosphorothioates and their 2′-O-allyl analogs and 2′-O-methylribonucleotide methylphosphonates which may be used in a nucleotide of the disclosure.

As used herein, the term “IRNA”, “RNAi”, “interfering nucleic acid” or “interfering RNA” means any nucleic acid, preferably RNA which is capable of down-regulating the expression of the targeted protein. Nucleic acid molecule interference designates a phenomenon by which dsRNA specifically suppresses expression of a target gene at post-transcriptional level. In normal conditions, RNA interference is initiated by double-stranded RNA molecules (dsRNA) of several thousands of base pair length. In vivo, dsRNA introduced into a cell is cleaved into a mixture of short dsRNA molecules called siRNA. The enzyme that catalyzes the cleavage, Dicer, is an endo-RNase that contains RNase III domains (Bernstein, Caudy et al. 2001 Nature. 2001 Jan. 18; 409(6818):363-6). In mammalian cells, the siRNAs produced by Dicer are 21-23 bp in length, with a 19 or 20 nucleotides duplex sequence, two-nucleotide 3′ overhangs and 5′-triphosphate extremities (Zamore, Tuschl et al. Cell. 2000 Mar. 31; 101(1):25-33; Elbashir, Lendeckel et al. Genes Dev. 2001 Jan. 15; 15(2): 188-200; Elbashir, Martinez et al. EMBO J. 2001 Dec. 3; 20(23):6877-88).

Said interfering nucleic acid can be as non-limiting examples anti-sense oligonucleotide constructs, small inhibitory RNAs (siRNAs) or short hairpin RNA.

Anti-sense oligonucleotides, including anti-sense RNA molecules and anti-sense DNA molecules, would act to directly block the translation of CILP-1 mRNA by binding thereto and thus preventing protein translation or increasing mRNA degradation, thus decreasing the level of CILP-1, and thus activity, in a cell. For example, antisense oligonucleotides of at least about 15 bases and complementary to unique regions of the mRNA transcript sequence can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In another embodiment, small inhibitory RNAs (siRNAs) can also be used to decrease the CILP-1 expression level in the present disclosure. CILP-1 gene expression can be reduced by administrating into a subject a small double stranded RNA (dsRNA), or a vector or construct causing the production of a small double stranded RNA, such that CILP-1 expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

In a preferred embodiment, short hairpin RNA (shRNA) can also be used to decrease the CILP-1 expression level in the present disclosure. A short hairpin RNA (shRNA) is a sequence of RNA that makes a tight hairpin turn that can be used to silence target gene expression via RNA interference (RNAi). Expression of shRNA in cells is typically accomplished by delivery of plasmids or through viral or bacterial vectors. The promoter choice is essential to achieve robust shRNA expression. At first, polymerase III promoters such as U6 and HI were used; however, these promoters lack spatial and temporal control. As such, there has been a shift to using polymerase II promoters to regulate expression of shRNA.

Interfering nucleic acid are usually designed against a region 19-50 nucleotides downstream the translation initiator codon, whereas 5′UTR (untranslated region) and 3′UTR are usually avoided. The chosen interfering nucleic acid target sequence should be subjected to a BLAST search against EST database to ensure that the only desired gene is targeted. Various products are commercially available to aid in the preparation and use of interfering nucleic acid.

In a particular embodiment, the interfering nucleic acid is a siRNA of at least about 10-40 nucleotides in length, preferably about 15-30 base nucleotides. In particular, interfering nucleic acid according to the disclosure comprises at least one sequence selected from the group consisting of:

(SEQ ID NO: 1)
5’-GCATGTGCCAGGACTTCATGC-3’
(SEQ ID NO: 2)
5’-GGTTCCGAGTTCCTGGCTTGT-3’
(SEQ ID NO: 3)
5’-GCCTGAAGTCAGCTACCATCA-3’
(SEQ ID NO: 4)
5’-GCTGGATCCCTCCCTCTATAA-3’

In a more preferred embodiment, up to four interfering nucleic acids comprising each a sequence SEQ ID NO: 1 to 4 are used concomitantly.

In a preferred embodiment, said interfering nucleic acid is a shRNA comprising at least one sequence selected from the group consisting of SEQ ID NO: 1 to 4, preferably comprising all the sequences SEQ ID NO: 1 to 4.

An interfering nucleic acid for use in the disclosure can be constructed using chemical synthesis and enzymatic ligation reactions using procedures known in the art. Particularly, interfering RNA can be chemically synthesized, produced by in vitro transcription from linear (e.g. PCR products) or circular templates (e.g., viral or non-viral vectors), or produced by in vivo transcription from viral or non-viral vectors. Interfering nucleic acid may be modified to have enhanced stability, nuclease resistance, target specificity and improved pharmacological properties. For example, antisense nucleic acid may include modified nucleotides or/and backbone designed to increase the physical stability of the duplex formed between the antisense and sense nucleic acids.

Small Molecules

In another particular embodiment, CILP-1 inhibitor can be a small molecule inhibiting the CILP-1 expression, activity or function.

As used herein, the term “small molecule inhibiting CILP-1 activity, expression or function” refers to small molecule that can be an organic or inorganic compound, usually less than 1000 daltons, with the ability to inhibit or reduce the activity, expression or function of CILP-1 protein. This small molecule can be derived from any known organism (including, but not limited to, animals, plants, bacteria, fungi and viruses) or from a library of synthetic molecules.

Small molecules inhibiting CILP-1 activity, expression or function can be identified by measuring the expression level of TGF-β or by measuring the expression level of CILP-1 as described above.

Nucleases

In another particular embodiment, CILP-1 inhibitor is a specific nuclease able to target and inactivate CILP-1 gene. Different types of nucleases can be used, such as Meganucleases, TAL-nucleases, zing-finger nucleases (ZFN), or RNA/DNA guided endonucleases like Cas9/CRISPR or Argonaute.

By “inactivating a target gene”, it is intended that the gene of interest is not or less expressed in a functional protein form. In particular embodiment, said nuclease specifically catalyzes cleavage in one targeted gene thereby inactivating said targeted gene.

The term “nuclease” refers to a wild type or variant enzyme capable of catalyzing the hydrolysis (cleavage) of bonds between nucleic acids within a DNA or RNA molecule, preferably a DNA molecule. In a particular embodiment, said nuclease according to the present disclosure is a RNA-guided endonuclease such as the Cas9/CRISPR complex. RNA guided endonucleases is a genome engineering tool where an endonuclease associates with a RNA molecule. In this system, the RNA molecule nucleotide sequence determines the target specificity and activates the endonuclease (Gasiunas, Barrangou et al. 2012; Jinek, Chylinski et al. 2012; Cong, Ran et al. 2013; Mali, Yang et al. 2013). Cas9/CRISPR involves a Cas9 nuclease and a guide RNA, also referred here as single guide RNA. Said single guide RNA is preferably able to target CILP-1 gene.

The inactivation of said target gene can also be performed by the use of site-specific base editors, for example by introducing premature stop codon(s), deleting a start codon or altering RNA splicing. Base editing directly generates precise point mutations in DNA without creating DNA double strand breaks. In a particular embodiment, base editing is performed by using DNA base editors which comprise fusions between a catalytically impaired Cas nuclease and a base modification enzyme that operates on single-stranded DNA (for review, see Rees H. A. et al. Nat Rev Genet. 2018. 19(12):770-788.

Nucleic Acid Construct

In a preferred embodiment, said nuclease, guide RNA, anti-sense oligonucleotide constructs, small inhibitory RNAs (siRNAs) or short hairpin RNA is included in a nucleic acid construct coding for them.

The term “nucleic acid construct” as used herein refers to a man-made nucleic acid molecule resulting from the use of recombinant DNA technology. A nucleic acid construct is a nucleic acid molecule, either single- or double-stranded, which has been modified to contain segments of nucleic acids sequences, which are combined and juxtaposed in a manner, which would not otherwise exist in nature. A nucleic acid construct usually is a “vector”, i.e. a nucleic acid molecule which is used to deliver exogenously created DNA into a host cell.

Preferably, the nucleic acid construct comprise said nuclease, guide RNA, anti-sense oligonucleotide constructs, small inhibitory RNAs (siRNAs) or short hairpin RNA operably linked to one or more control sequences that direct the expression in cardiac cells.

In a preferred embodiment, said nucleic acid construct comprises an interfering nucleic acid able to repress CILP-1 gene expression comprising at least one sequence selected from sequences SEQ ID NO: 1 to 4. More preferably, said nucleic acid construct comprises four interfering nucleic acid of sequences SEQ ID NO: 1 to 4.

Expression Vector

The nucleic acid construct as described above may be contained in an expression vector. The vector may be an autonomously replicating vector, i.e., a vector that exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated.

Examples of appropriate vectors include, but are not limited to, recombinant integrating or non-integrating viral vectors and vectors derived from recombinant bacteriophage DNA, plasmid DNA or cosmid DNA. Preferably, the vector is a recombinant integrating or non-integrating viral vector. Examples of recombinant viral vectors include, but not limited to, vectors derived from herpes virus, retroviruses, lentivirus, vaccinia viruses, adenoviruses, adeno-associated viruses or bovine papilloma virus.

AAV has arisen considerable interest as a potential vector for human gene therapy. Among the favourable properties of the virus are its lack of association with any human disease, its ability to infect both dividing and non-dividing cells, and the wide range of cell lines derived from different tissues that can be infected.

The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length. The internal non-repeated portion of the genome includes two large open reading frames, known as the AAV rep and cap genes, respectively. These genes code for the viral proteins involved in replication and packaging of the virion. In particular, at least four viral proteins are synthesized from the AAV rep gene, Rep 78, Rep 68, Rep 52 and Rep 40, named according to their apparent molecular weight. The AAV cap gene encodes at least three proteins, VP1, VP2 and VP3. For a detailed description of the AAV genome, see, e.g., Muzyczka, N. 1992 Current Topics in Microbiol. and Immunol. 158:97-129.

Thus, in one embodiment, the nucleic acid construct or expression vector comprising transgene as described above further comprises a 5′ITR and a 3′ITR sequences, preferably a 5′ITR and a 3′ ITR sequences of an adeno-associated virus.

As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles.

AAV ITRs for use in the viral vector of the disclosure may have a wild-type nucleotide sequence or may be altered by the insertion, deletion or substitution. The serotype of the inverted terminal repeats (ITRs) of the AAV may be selected from any known human or nonhuman AAV serotype. In specific embodiments, the nucleic acid construct or viral expression vector may be carried out by using ITRs of any AAV serotype, including AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV, and any other AAV serotype or engineered AAV now known or later discovered.

In one embodiment, the nucleic acid construct further comprises a 5′ITR and a 3′ITR of the corresponding capsid, or preferably 5′ITR and a 3′ITR of a serotype AAV-2.

On the other hand, the nucleic acid construct or expression vector of the disclosure can be carried out by using synthetic 5′ITR and/or 3′ITR; and also by using a 5′ITR and a 3′ITR which come from viruses of different serotypes. All other viral genes required for viral vector replication can be provided in trans within the virus-producing cells (packaging cells) as described below. Therefore, their inclusion in the viral vector is optional.

In one embodiment, the nucleic acid construct or viral vector of the disclosure comprises a 5′ITR, a ψ packaging signal, and a 3′ITR of a virus. “ψ packaging signal” is a cis-acting nucleotide sequence of the virus genome, which in some viruses (e.g. adenoviruses, lentiviruses . . . ) is essential for the process of packaging the virus genome into the viral capsid during replication.

The construction of recombinant AAV viral particles is generally known in the art and has been described for instance in U.S. Pat. Nos. 5,173,414 and 5,139,941; WO 92/01070, WO 93/03769, Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801.

Viral Particle

In a preferred embodiment, the present disclosure relates to viral particles including a nucleic acid construct or expression vector as described above.

The nucleic acid construct or the expression vector of the disclosure may be packaged into a virus capsid to generate a “viral particle”, also named “viral vector particle”. In a particular embodiment, the nucleic acid construct or the expression vector as described above is packaged into an AAV-derived capsid to generate an “adeno-associated viral particle” or “AAV particle”. The present disclosure relates to a viral particle comprising a nucleic acid construct or an expression vector of the disclosure and preferably comprising capsid proteins of adeno-associated virus.

The term AAV vector particle encompasses any recombinant AAV vector particle or mutant AAV vector particle, genetically engineered. A recombinant AAV particle may be prepared by encapsidating the nucleic acid construct or viral expression vector including ITR(s) derived from a particular AAV serotype on a viral particle formed by natural or mutant Cap proteins corresponding to an AAV of the same or different serotype.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

Several techniques have been developed to modify and improve the structural and functional properties of naturally occurring AAV viral particles (Bunning H et al. J Gene Med, 2008; 10: 717-733; Paulk et al. Mol ther. 2018; 26(1):289-303; Wang L et al. Mol Ther. 2015; 23(12):1877-87; Vercauteren et al. Mol Ther. 2016; 24(6):1042-1049; Zinn E et al., Cell Rep. 2015; 12(6):1056-68).

Thus, in AAV viral particle according to the present disclosure, the nucleic acid construct or viral expression vector including ITR(s) of a given AAV serotype can be packaged, for example, into: a) a viral particle constituted of capsid proteins derived from the same or different AAV serotype; b) a mosaic viral particle constituted of a mixture of capsid proteins from different AAV serotypes or mutants; c) a chimeric viral particle constituted of capsid proteins that have been truncated by domain swapping between different AAV serotypes or variants.

The skilled person will appreciate that the AAV viral particle for use according to the present disclosure may comprise capsid proteins from any AAV serotype including AAV1, AAV2, AAV3 (including types 3A and 3B), AAV4, AAV5, AAV6, AAV7, AAV8, AAV9, AAV10, AAV11, AAV12, AAV2i8, AAVrh10, AAVrh39, AAVrh43, AAVrh74, AAV-LK03, AAV2G9, AAV.PHP, AAV-Anc80, AAV3B and AAV9.rh74 (as disclosed in WO2019/193119).

For gene transfer into human cardiac cells, AAV serotypes 1, 6, 8, 9 and AAV9.rh74 are preferred. The AAV serotype 9 and AAV9.rh74 are particularly well suited for the induction of expression in cells of the myocardium/cardiomyocytes. In a specific embodiment, the AAV viral particle comprises a nucleic acid construct or expression vector of the disclosure and preferably capsid proteins from AAV9 or AAV9.rh74 serotype.

Pharmaceutical Composition

The CILP-1 inhibitor, nucleic acid construct, expression vector or viral particle according to the present disclosure is preferably used in the form of a pharmaceutical composition comprising a therapeutically effective amount of CILP-1 inhibitor, nucleic acid construct, expression vector or viral particle according to the present disclosure.

In the context of the disclosure, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies.

The term “effective dose” or “effective dosage” is defined as an amount sufficient to achieve, or at least partially achieve, the desired effect.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present disclosure, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

Treatment of Dilated Cardiomyopathies

The CILP-1 inhibitor, nucleic acid construct or viral particle according to the present disclosure is used for the treatment of any dilated cardiomyopathy (DCM).

Dilated cardiomyopathy (CMD) is characterized by cardiac dilatation and reduced systolic function. CMD is the most frequent form of cardiomyopathy and accounts for more than half of all cardiac transplantations performed in patients between 1 and 10 years of age. Causes of DCMs include in particular genetics, and a variety of toxic, metabolic or infectious agents. Toxic or metabolic agents include in particular alcohol and cocaine abuse and chemotherapeutic agents such as for example doxorubicin and cobalt; Thyroid disease; inflammatory diseases such as sarcoidosis and connective tissue diseases; Tachycardia-induced cardiomyopathy; autoimmune mechanisms; complications of pregnancy; and thiamine deficiency. Infectious agents include in particular Chagas disease due to Trypanosoma cruzi and sequelae of acute viral myocarditis such as for example with Coxsackie B virus and other enteroviruses. A heritable pattern is present in 20 to 30% of cases. Most familial CMD pedigrees show an autosomal dominant pattern of inheritance, usually presenting in the second or third decade of life (summary by Levitas et al., Europ. J. Hum. Genet., 2010, 18: 1160-1165).

In genetically-induced dilated cardiomyopathies, most of the genes involved code for structural elements of cardiomyocytes, including extracellular matrix or Golgi apparatus proteins (laminin, fukutin) involved in cellular adhesion and signaling pathways; desmosome proteins (desmocollin, plakoglobin) involved in cellular junctions; sarcoplasmic reticulum proteins (RyR2, SERCA2a, phospholamban) involved in calcium homeostasis; nuclear envelop proteins (lamin A/C) involved in myocardial structural organisation; cytoskeleton proteins (dystrophin, telethonin, α-actinin, desmin, sarcoglycans) involved in cytoskeleton integrity and muscular strength transmission; and sarcomer proteins (titin, troponin, myosin, actin) involved in generation and transmission of muscular strength.

Mutations in many genes have been found to cause different forms of dilated cardiomyopathy (CMD). These include in particular:
CMD1A, dilated cardiomyopathy-1A (OMIM #115200) caused by heterozygous mutation in the lamin A/C gene (LMNA), (OMIM #150330) on chromosome 1q22; or heterozygous mutation in the laminin alpha 2 (LAMA2 or MEROSIN) gene (OMIM #156225; Marques et al., Neuromuscul. Disord., 2014, doi.org/10.10106/);
CMD1B (OMIM #600884) on 9q13; the gene referred as FDC locus was placed in the interval between D9S153 and D9S152. Friedreich ataxia (OMIM #229300), which is frequently associated with dilated cardiomyopathy, maps to the same region as does also the cAMP-dependent protein kinase (OMIM #176893), which regulates calcium-channel ion conductance in the heart. Tropomodulin (OMIM #190930), which maps to 9q22, was a particularly attractive candidate gene.
CMD1C (OMIM #601493) with or without left ventricular noncompaction, caused by mutation in the lim domain-binding 3, LDB3 (or ZASP) gene (OMIM #605906) on 10q23;
CMD1D (OMIM #601494), caused by mutation in the troponin T2, cardiac (TNNT2) gene (OMIM #191045) on 1q32;
CMD1E (OMIM #601154), caused by mutation in the SCN5A gene (OMIM #600163) on 3p22;
CMD1F: The symbol CMD1F was formerly used for a disorder later found to be the same as desmin-related myopathy or myopathy, myofibrillar (MFM) (OMIM #601419);
CMD1G (OMIM #604145), caused by mutation in the titin (TTN) gene (OMIM #188840) on 2q31;
CMD1H (OMIM #604288) on 2q14-q22;
CMD1I (OMIM #604765), caused by mutation in the desmin (DES) gene (OMIM #125660) on 2q35;
CMD1J (OMIM #605362), caused by mutation in the EYA4 gene (OMIM #603550) on 6q23;
CMD1K (OMIM #605582) on 6q12-q16;
CMD1L (OMIM #606685), caused by mutation in the sarcoglycan delta (SGCD) gene (OMIM #601411) on 5q33;
CMD1M (OMIM #607482), caused by mutation in the CSRP3 gene (OMIM #600824) on 11p15;
CMD1N; (OMIM #607487) caused by mutation in the TITIN-CAP (telethonin or TCAP) gene (OMIM #604488).
CMD1O (OMIM #608569), caused by mutation in the ABCC9 gene (OMIM #601439) on 12p12;
CMD1P (OMIM #609909), caused by mutation in the phospholamban (PLN) gene (OMIM #172405) on 6q22;
CMD1Q (OMIM #609915) on 7q22.3-q31.1;
CMD1R (OMIM #613424), caused by mutation in the actin alpha, cardiac muscle (ACTC1) gene (OMIM #102540) on 15q14;
CMD1S (OMIM #613426), caused by mutation in the myosin heavy chain 7, cardiac muscle, beta (MYH7) gene (OMIM #160760) on 14q12;
CMD1U (OMIM #613694), caused by mutation in the PSEN1 gene (OMIM #104311) on 14q24;
CMD1V (OMIM #613697), caused by mutation in the PSEN2 gene (OMIM #600759) on 1q42;
CMD1W (OMIM #611407), caused by mutation in the gene encoding metavinculin (VCL; OMIM #193065) on 10q22;
CMD1X (OMIM #611615), caused by mutation in the gene encoding fukutin (FKTN; OMIM #607440) on 9q31;
CMD1Y (OMIM #611878), caused by mutation in the TPM1 gene (OMIM #191010) on 15q22;
CMD1Z (OMIM #611879), caused by mutation in the troponin C (TNNC1) gene (OMIM #191040) on 3p21;
CMD1AA (OMIM #612158), caused by mutation in the actinin alpha-2 (ACTN2) gene (OMIM #102573) on 1q43;
CMD1BB (OMIM #612877), caused by mutation in the DSG2 gene (OMIM #125671) on 18q12;
CMD1CC (OMIM #613122), caused by mutation in the NEXN gene (OMIM #613121) on 1p31;
CMD1DD (OMIM #613172), caused by mutation in the RNA binding motif protein 20 (RBM20) gene (OMIM #613171) on 10q25;
CMD1EE (OMIM #613252), caused by mutation in the myosin heavy chain 6, cardiac muscle, alpha (MYH6) gene (OMIM #160710) on 14q12;
CMD1FF (OMIM #613286), caused by mutation in the troponin I, cardiac (TNNI3) gene (OMIM #191044) on 19q13;
CMD1GG (OMIM #613642), caused by mutation in the SDHA gene (OMIM #600857) on 5p15;
CMD1HH (OMIM #613881), caused by mutation in the BCL2-associated athanogene 3 (BAGS) gene (OMIM #603883) on 10q26;
CMD1II (OMIM #615184), caused by mutation in the CRYAB gene (OMIM #123590) on 6q21;
CMD1JJ (OMIM #615235), caused by mutation in the laminin alpha 4 (LAMA4) gene (OMIM #600133) on 6q21;
CMD1KK (OMIM #615248), caused by mutation in the MYPN gene (OMIM #608517) on 10q21;
CMD1LL (OMIM #615373), caused by mutation in the PRDM16 gene (OMIM #605557) on 1p36;
CMD1MM (OMIM #615396), caused by mutation in the MYBPC3 gene (OMIM #600958) on 11p11;
CMD1NN (OMIM #615916), caused by mutation in the RAF1 gene (OMIM #164760) on 3p25;
CMD2A (OMIM #611880), caused by mutation in the troponin I, cardiac (TNNI3) gene on 19q13;
CMD2B (OMIM #614672), caused by mutation in the GATAD1 gene (OMIM #614518) on 7q21;
CMD2C (OMIM #618189), caused by mutation in the PPCS gene (OMIM #609853) on 1p34;
CMD3A, a previously designated X-linked form was found to be the same as Barth syndrome (OMIM #302060); and
CMD3B (OMIM #302045), an X-linked form of CMD, caused by mutation in the dystrophin gene (DMD, OMIM #300377).

Desmin-related myopathy or myopathy, myofibrillar (MFM) (OMIM #601419). is a noncommittal term that refers to a group of morphologically homogeneous, but genetically heterogeneous chronic neuromuscular disorders. The morphologic changes in skeletal muscle in MFM result from disintegration of the sarcomeric Z disc and the myofibrils, followed by abnormal ectopic accumulation of multiple proteins involved in the structure of the Z disc, including desmin, alpha-B-crystallin (CRYAB; OMIM #123590), dystrophin (OMIM #300377), and myotilin (TTID; OMIM #604103). Myofibrillar myopathy-1 (MFM1) is caused by heterozygous, homozygous, or compound heterozygous mutation in the desmin gene (DES; OMIM #125660) on chromosome 2q35. Other forms of MFM include MFM2 (OMIM #608810), caused by mutation in the CRYAB gene (OMIM #123590); MFM3 (OMIM #609200) (OMIM #182920), caused by mutation in the MYOT gene (OMIM #604103); MFM4 (OMIM #609452), caused by mutation in the ZASP gene (LDB3; OMIM #605906); MFM5 (OMIM #609524), caused by mutation in the FLNC gene (OMIM #102565); MFM6 (OMIM #612954), caused by mutation in the BAGS gene (OMIM #603883); MFM7 (OMIM #617114), caused by mutation in the KY gene (OMIM #605739); MFM8 (OMIM #617258), caused by mutation in the PYROXD1 gene OMIM #617220); and MFM9 (OMIM #603689), caused by mutation in the TTN gene (titin; OMIM #188840).

Mutations in other genes have also been found to cause different forms of dilated cardiomyopathy. These include:
desmocollin 2 (DSC2, OMIM #125645) responsible for Arrhythmogenic Right Ventricular Dysplasia 11 (OMIM #610476) and dilated cardiomyopathy (Elliott et al., Circ. Vasc. Genet., 2010, 3, 314-322);
junctiun plakoglobin (JUP or plakoglobin; OMIM #173325) responsible for Arrhythmogenic Right Ventricular Dysplasia 12 (OMIM #611528) and dilated cardiomyopathy (Elliott et al., Circ. Vasc. Genet., 2010, 3, 314-322);
ryanodine receptor 2 (RYR2; OMIM #180902) responsible for Arrhythmogenic Right Ventricular Dysplasia 2 (OMIM #600996) and Ventricular tachycardia, catecholaminergic polymorphic 1 (OMIM #604772) and dilated cardiomyopathy (Zahurul, Circulation, 2007, 116, 1569-1576);
ATPase, Ca(2+)-transporting slow-twitch (ATP2A2; ATP2B, sarcoplasmic reticulum Ca(2+) ATPase 2 isoform alpha (SERCA2a); and
emerin (EMD); fukutin-related protein (FKRP); tafazzin (TAZ); desmoplakin (DSP); and Sodium Channels such as SCN1B, SCN2B, SCN3B, SCN4B, SCN4A, SCN5A and others.

In some embodiments, the dilated cardiomyopathy is an acquired dilated cardiomyopathy; for example caused by toxic, metabolic or infectious agents according to the present disclosure. The cause of the dilated cardiomyopathy may also be unknown (idiopathic dilated cardiomyopathy).

In some preferred embodiments, the dilated cardiomyopathy is a genetic dilated cardiomyopathy; preferably caused by mutation(s) in a gene selected from the group consisting of: laminin, in particular laminin alpha 2 (LAMA2) and laminin alpha 4 (LAMA4); emerin (EMD); fukutin (FKTN); fukutin-related protein (FKRP); desmocollin, in particular desmocollin 2 (DSC2); plakoglobin (JUP); ryanodine receptor 2 (RYR2); sarcoplasmic reticulum Ca(2+) ATPase 2 isoform alpha (SERCA2a); phospholamban (PLN); lamin A/C (LMNA); dystrophin (DMD); TITIN-CAP or telethonin (TCAP); actinin, in particular actinin alpha-2 (ACTN2); desmin (DES); actin, in particular cardiac actin, actin alpha, cardiac muscle (ACTC1); sarcoglycans, in particular sarcoglycan delta (SGCD); titin (TTN); troponin, in particular cardiac troponin, troponin T2, cardiac (TNNT2); troponin C (TNNC1) and troponin I, cardiac (TNNI3); myosin, in particular myosin heavy chain 7, cardiac muscle, beta (MYH7) and myosin heavy chain 6, cardiac muscle, alpha (MYH6); RNA binding motif protein 20 (RBM20); BCL2-associated athanogene 3 (BAG3); desmoplakin (DSP); tafazzin (TAZ) and sodium channels such as SCN1B, SCN2B, SCN3B, SCN4B, SCN4A, SCN5A and others, preferably dystrophin (DMD) or titin (TTN).

The disclosure provides also a method for treating a dilated cardiomyopathy according to the present disclosure, comprising: administering to a patient a therapeutically effective amount of the CILP-1 inhibitor, nucleic acid construct or viral particle or pharmaceutical composition as described above.

The disclosure provides also the use of the CILP-1 inhibitor, nucleic acid construct or viral particle or pharmaceutical composition as described above for the treatment of a dilated cardiomyopathy according to the present disclosure.

The disclosure provides also the use of the CILP-1 inhibitor, nucleic acid construct or viral particle or pharmaceutical composition as described above in the manufacture of a medicament for treatment of a dilated cardiomyopathy according to the present disclosure.

The disclosure provides also a pharmaceutical composition comprising the CILP-1 inhibitor, nucleic acid construct or viral particle or as described above for treating a dilated cardiomyopathy according to the present disclosure

The disclosure provides also a pharmaceutical composition for treatment of a dilated cardiomyopathy according to the present disclosure, comprising the CILP-1 inhibitor, nucleic acid construct or viral particle or as described above as an active component.

As used herein, the term “patient” or “individual” denotes a mammal. Preferably, a patient or individual according to the disclosure is a human.

In the context of the disclosure, the term “treating” or “treatment”, as used herein, means reversing, alleviating or inhibiting the progress of the disorder or condition to which such term applies, or reversing, alleviating or inhibiting the progress of one or more symptoms of the disorder or condition to which such term applies.

The pharmaceutical composition of the present disclosure is generally administered according to known procedures, at dosages and for periods of time effective to induce a therapeutic effect in the patient.

The administration can be systemic, local or systemic combined with local. Systemic administration is preferably parenteral such as subcutaneous (SC), intramuscular (IM), intravascular such as intravenous (IV) or intraarterial; intraperitoneal (IP); intradermal (ID) or else. Local administration is preferably intracerebral, intracerebroventricular, intracisternal, and/or intrathecal administration. The administration may be for example by injection or perfusion. In some preferred embodiments, the administration is parenteral, preferably intravascular such as intravenous (IV) or intraarterial. In some other preferred embodiments, the administration is intracerebral, intracerebroventricular, intracisternal, and/or intrathecal administration, alone or combined with parenteral administration, preferably intravascular administration. In some other preferred embodiments, the administration is parenteral, preferably intravascular alone or combined with intracerebral, intracerebroventricular, intracisternal, and/or intrathecal administration. The practice of the present disclosure will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1: Map of shRNA 4 in 1 mCILP-GFP plasmid.

FIG. 2: qPCR analysis of CLP-1 gene in the RNAseq. n=4 per group. Student test.

FIG. 3: Expression of transgenes. Relative RT-qPCR abundance of the GFP transgene in DeltaMex5 mice injected or not injected by the vectors AAV9-4in1shRNA-mCILP-GFP. n=4. Student's test.

FIG. 4: Morphological analysis. A) Total mass of mice. B) Measurement of cardiac hypertrophy: heart mass/total mouse mass (%).

FIG. 5: Histological characterization of the heart after injection of AAV9-shCILP in DeltaMex5 mice and controls injected with PBS. A) HPS staining of the heart. B) Sirius red staining of the heart. Scale, 500 μm.

FIG. 6: Comparison of one DCM marker (left ventricular mass) measured in ultrasound between C57BL/6 mice, DeltaMex5 mice and DeltaMex5 mice injected with shCILP vector. Student test.

FIG. 7: RT-qPCR measurement of different RNA markers of cardiac involvement. Measurements expressed as a ratio to the C57BL/6 mouse. Student's test.

FIG. 8: RT-qPCR measurement of various RNA markers of cardiac fibrosis. Measurements expressed as a ratio to the C57BL/6 mouse. Student's test.

### EXAMPLES

1. Material and Methods

1.1 Mouse Models

The mice used in this study were male titinMex5−/Mex5− (DeltaMex5) and DBA/2J-mdx (DBA2mdx) strains, and their respective controls, strains C57BL/6 and DBA/2. DeltaMex5 mice are mice in which the penultimate exon of Titin is deleted by CRISPR-Cas9 technology (Charton, K., et al. 2016, Human molecular genetics 25, 4518-4532). DBA2mdx mice are a model of Duchenne muscular dystrophy due to a point mutation on exon 23 of the dystrophin gene. DBA2mdx mice are on a DBA/J background which has a mutation on the LTBP4 gene, a protein that regulates the activity of the TGF signalling pathway-β (Fukada, et al. 2010. Am J Pathol 176, 2414-2424). All the mice are handled in accordance with the European directives for the care and use of laboratory animals by humans, and the animal experimentation has been approved by the Ethics Committee for Animal Experimentation C2AE-51 of Evry under the numbers of Project Authorisation Application 2015-003-A and 2018-024-B.

1.2 Muscle Sampling and Freezing

The muscles of interest are collected, weighed and frozen in liquid nitrogen (samples for molecular biology analysis) or in cooled isopentane (samples for histology), after being placed transversely or longitudinally on a piece of cork coated with gum arabic. The hearts are frozen in diastole before being frozen with a diluted butanedione solution (5 mM) in tyrode. The samples are then stored at −80° C. until use. For the Sirius Red Fibrosis observation protocol on the whole heart, the hearts are included whole in paraffin and stored at room temperature. For the transparency protocols, the sampled hearts are stored whole in 4% para formaldehyde and kept at +4° C.

1.3 Haematoxylin-Phloxine-Safran Staining

The Hematoxylin-Phloxine-Safran (HPS) marking allows to observe the general appearance of the muscle and to highlight the different tissue and cell structures. Haematoxylin colours nucleic acids dark blue, phloxine colours the cytoplasm pink, saffron colours collagen red-orange. Cross sections are stained with Harris hematoxylin (Sigma) for 5 min. After washing with water for 2 min, the slides are immersed in a 0.2% (v/v) hydrochloric alcohol solution for 10 s to remove excess stain. After being washed again with water for 1 min, the tissues are blued in a Scott water bath (0.5 g/l sodium bicarbonate and 20 g/l magnesium sulfate solution) for 1 min before being rinsed again with water for 1 min and stained with phloxine 1% (w/v) (Sigma) for 30 s. After rinsing with water for 1 min 30 s, the cuts are dehydrated with 70° ethanol for 1 min and then rinsed in absolute ethanol for 30 s. The tissues are then stained with saffron 1% (v/v in absolute ethanol) for 3 min and rinsed in absolute ethanol. Finally, the cuts are thinned in a Xylene bath for 2 min and then mounted with a slide in the Eukitt medium. Image acquisition is performed with objective 10 on a Zeiss AxioScan white light microscope coupled to a computer and a motorized stage.

From HPS coloured sections, the centronucleation index is calculated by the ratio of the number of centronucleated fibres to the area of the section in mm2.

1.4 Sirius Red Coloration

Sirius Red staining allows the collagen fibres to be coloured red and to highlight the presence of fibrotic tissue. Cytoplasms are stained yellow. Cross sections are dehydrated with acetone for 1 hour for frozen cuts or dewaxed with heat and toluene baths. They are then fixed with 4% formaldehyde for 5 min then 10 min in a Bouin solution. After two washes with water, the slides are immersed in Sirius Red solution (0.1 g Sirius Red per 100 mL picric acid solution) for 1 h for staining. After rinsing with water for 1 min 30 s, the slices are dehydrated in successive ethanol baths: 70° ethanol for 1 min, 95° ethanol for 1 min, absolute ethanol for 1 min and then a second absolute ethanol bath for 2 min. Finally, the slices are thinned in two Xylene baths for 1 min and then mounted with a lamella in the Eukitt medium. Image acquisition is carried out with objective 10× on a Zeiss AxioScan white light microscope coupled to a computer and a motorized stage. The polarized light images were acquired using a modified right LEICA microscope.

1.5 Quantification of Sirius Red

Sirius Quant

Sirius Quant is an internally developed ImageJ pluggin (Schneider et al., 2012). It is a thresholding macro that allows to isolate and quantify the pixels of the image that are colored red. It works in 3 steps: the first one is to convert the image to black and white. The images resulting from the Red Sirius colorations are very contrasted, so a simple black and white conversion is enough to keep all the useful information. The second one is a very rough thresholding in order to keep only the colored pixels of the image, in other words the pixels belonging to the whole cut. Using the Analyze Particles function with an adapted object size allows automatic detection of the outline of the slice, which is then stored. The third step is a manual thresholding by the user which allows to keep only the pixels colored in red, those associated with the marking. A manual correction tool makes it possible either to remove areas that would have been detected and that are not marking (dust, cut fold, etc.), or to add areas that would not have been taken into account. Once the thresholded image is satisfactory, the number of thresholded pixels and the total number of pixels in the entire section are then measured. A ratio between these two numbers finally gives the fibrosis index in the slice.

WEKA

The images were processed using an artificial intelligence algorithm via the WEKA plugin (ImageJ). The WEKA classifier pluggin was implemented using a training data set containing 17 images representative of the different conditions to be classified. The classes were assigned to healthy tissue (yellow), to both types of staining and to slice rupture (white). The original mappings are mosaic images with a size of approximately 225 megapixels (15 k×15 k), which were divided into 400 frames (20 rows, 20 columns), each frame measuring approximately 750×750 pixels. Each frame is classified independently and the complete image is then reconstructed. The number of pixels in each class is measured. The total number of pixels belonging to the heart is calculated as the sum of the healthy tissue and the two types of dye uptake. The ratio of each class is then calculated by dividing the number of pixels in the class by the total number of pixels in the heart.

Whole-Heart Reconstruction and Quantification

Sections of a whole heart colored by Sirius Red were scanned with a scanner (Axioscan ZI, Zeiss) with a 10×lens. A total of 483 images were obtained. They were aligned using Imagers pluggin: Linear Stack Alignment with SIFT (Lowe et al., International Journal of Computer Vision, 2004, 60, 91-110). Some images were manually aligned when the software did not allow a satisfactory alignment. The image was loaded into Imaris (BitPlane, USA) for reconstruction and 3D visualization. Once the images were aligned, the Sirius Quant pluggin in fully automatic mode using Otsu thresholding (Otsu N, Cybernetics, 1979, 9, 62-66) resulted in 483 fibrosis ratio values corresponding to each image. These values were filtered using the sliding average method, which is a method of reducing noise in a signal to avoid the errors inherent in automating an algorithm. The use of the moving average allows to limit these errors by replacing each fibrosis ratio of an image by the average of itself, the ratio of the image preceding it and the ratio of the image following it.

1.6 Fluorescence Immuno-Histo Labeling

The slides are taken out of the freezer and allowed to dry at room temperature for 10 minutes, after which the cuts are wrapped with DAKOpen. The slices are then rehydrated for 5 min in PBS 1×. If the protein of interest is located in the nucleus, the slices are permeabilised for 15 min in a 0,3% triton solution in PBS 1×, then washed 3 times in PBS for 5 min. The slices are then saturated with 10% goat serum, 10% fetal calf serum, PBS 1× for 30 min at room temperature in a humidity chamber. The saturation medium is replaced by the primary antibody solution diluted in PBS 1×+10% blocking solution overnight at 4° C. in a wet chamber. Four successive washes in 1× PBS for 5 minutes are performed before hybridizing with the secondary antibody solution coupled with an Alexa 488 or 594 (1/1000) fluorochrome coupled fluorochrome in 1× PBS+10% blocking solution for 1 h at room temperature in a light-protected wet chamber. A final series of four 5-minute washes in PBS 1× is performed and a fluoromount slide assembly containing DAPI is performed. The sections are then visualized using a fluorescence microscope (Zeiss AxioScan or Leica TCS-SP8 confocal microscope).

TABLE 1
List of antibodies used in immunohistology

| Antibody | Species | Supplier | Reference | Dilution |
|---|---|---|---|---|
| Collagen I | Mouse | Abeam | ab6308 | 1/100 |
| Collagen III | Rabbit | Abeam | ab7778 | 1/100 |
| Fibronectin | Mouse | Sigma | F7387 | 1/200 |
| Vimentine | Mouse | Chemicon | MAB3400 | 1/100 |
| Vinculine | Mouse | Sigma | V9131 | 1/100 |
| Actin F | Mouse | Abeam | ab205 | 1/100 |
| Titine N2B | Rabbit | Myomedix | #6678 | 1/75 |
| Titanium | Rabbit | Myomedix | #3375 | 1/75 |
| M8M9 | | | | |
| Titanium IS7-1 | Rabbit | Genescript | LVEEPPPREVVLKTSC | 1/2 |
| | | | (SEQ ID NO: 5) | |
| M10-1 | Rabbit | Genescript | IEALPSDISIDEGKV | 1/75 |
| Titanium | | | (SEQ ID NO: 6) | |
| α-synemin | Rabbit | SantaCruz | sc-68849 | 1/25 |
| Obscurine | Rabbit | Atlas | HPA040066 | 1/50 |
| | | Antibody | | |
| Myosprin | Rabbit | Abeam | ab75351 | 1/25 |
| Cilp | Rabbit | biorbyt | orb182643 | 1/100 |

1.7 RNA Extraction and Quantification

Frozen isopentane muscle is cut into 30 μm thick slices on a cryostat (LEICA CM 3050) at −20° C., separated into eppendorf tubes of about 10-15 slices and stored at −80° C. The TRIzol® method for the extraction of total RNA, based on the solvency properties of nucleic acids in organic solvents, is used.

The muscle recovery tubes are resupplied with 0.8 mL of TRIzol® (ThermoFisher) supplemented with glycogen (Roche) at a rate of 0.5 μL/mL of TRIzol®. The tubes are placed in the FastPrep-24 (Millipore) homogenizer for a 20 s, 4 m.s. cycle. To recover nucleic acids, after a 5-minute incubation on ice, 0.2 mL of chloroform (Prolabo) is added and mixed with TRIzol®. After incubation for 3 minutes at room temperature, the two phases, aqueous and organic, are separated by centrifugation at 12000 g for 15 minutes at 4° C. The aqueous phase, containing the nucleic acids, is removed and placed in a new tube. The RNAs are then precipitated by the addition of 0.5 mL isopropanol (Prolabo) followed by a incubation for 10 minutes at room temperature and centrifugation for 15 minutes at 12000 g at 4° C. The nucleic acid pellet is washed with 0.5 mL 75% ethanol (Prolabo) and again centrifuged for 10 minutes at 12000 g at 4° C. and then air-dried. The nucleic acids are taken up in 50 μL of nuclease free water, 20 μL are set aside for viral DNA analysis, 30 μL are added to RNAsin (Promega) diluted at 1/50 to preserve the RNAs from degradation. The RNAs are then treated with TURBO Dnase (Ambion) to remove residual DNA. A double Dnase treatment is performed for samples intended for sequencing.

For transcriptome analysis specific to signaling pathways, RT2 Profiler PCR Array (Qiagen) plates are used. The screening plates require the use of a compatible RNA extraction kit, the RNeasy Mini Kit (Qiagen) which extracts the RNA on columns, the kit is used following the supplier's instructions, and the RNAs are then processed by Free DNAse RNAse (Qiagen).

An OD reading is then taken on the ND-8000 spectrophotometer (Nanodrop), from 2 μL of RNA to determine their concentration. RNA is stored at −80° C. and DNA at −20° C.

1.8 Measurement of RNA Quality

In the case of RNAs prepared for sequencing, the quality of the RNAs is measured on the Bioanalyzer 2100 (Agilent) which performs capillary electrophoresis of nucleic acids and then their analysis. The quality is visualized by the retention rate and the concentration of the sample in the form of electrophoregrams. A quality score expressed in RIN (for RNA Integrity Number) is calculated for each sample, on a scale of 0 to 10. The RNA Nano chip (Agilent) is used according to the supplier's instructions. A size marker (RNA 6000 Nano Ladder, Agilent) is passed first, to allow evaluation of RNA size in the samples. A marker is added to each sample, emerging at a defined size. For each sample, 1 μL of RNA is deposited on the chip. On the RNA electrophoregram, the ribosomal RNA peaks are observed: 28S (around 4000 nt), 18S (around 2000 nt) and 5S (around 100 nt). The internal marker emerges at the 25 nt position. The INR is calculated as a function of the height and position of the 18S and 28S peaks, the ratio between the 5S, 18S and 28S peaks, and the signal-to-noise ratio. For RNA-seq, the required quality requires an INR of at least 7.

1.9 Real-Time Quantitative PCR

Genomic and viral DNA are quantified by qPCR and gene expression by Real-time quantitative PCR. Reverse transcription step is performed on the entire messenger RNA using the RevertAid H Minus First Strand cDNA Synthesis Kit (Thermo-Fisher). Two types of oligonucleotides: so-called “random” hexamers, containing random sequences, and “dT” oligonucleotides, deoxy-thymine polymers, which hybridize to the polyA sequences, making it possible to generate cDNAs in their entirety. The mix used is shown in Table 2.

TABLE 2
Reaction mixture for reverse transcription

| Product | Quantity | |
|---|---|---|
| RNA | 1 | μg |
| Random hexameres + 1/10 | 50 | ng |
| OligodT | | |
| Reaction buffer 5X | ⅕ | |
| dNTP | 500 μM of each | |
| Ribolock Rnase Inhibitor 40 U/μl | 0.25 | U |
| RevertAid H-Minus 200 U/μL | 200 | U |

| | |
|---|---|
| Water | qsp 20 μL |

The mixture is placed in a thermal cycler for the following cycle: 10 min at 25° C., then 1 h15 at 42° C., temperature of action of the enzyme, then the enzyme is inactivated 10 min at 70° C. The cDNAs are stored at +4° C. in the short term or at −20° C. in the long term.

Real-time quantitative PCR is performed either on genomic or viral DNA for vector titration and measurement of vector copy number in tissues, or on cDNA obtained from RNA for quantification of transcripts. It is performed on the LightCycler 480® (Roche) 384-well plate. The nuclease activity of the Thermo-Start DNA Polymerase enzyme contained in AB solute QPCR ROX Mix (ThermoFisher) allows the detection of PCR products at each amplification cycle by release of a fluorescent reporter. This fluorescent reporter is a fluorophore (FAM, for 6-carboxyfluorescein or VIC, for 2′-chloro-7′phenyl-1,4-dichloro-6-carboxy-fluorescein) is located 5′ from the nucleotide probe which is also labelled with a quencher (TAMRA, for tetramethylrhodamine) in 3′. Separation of the reporter and the quencher results in the fluorescence of the reporter, which is measured by the apparatus. The mixtures for each gene of interest are composed of the two oligos F (forward, sense) and R (reverse, antisense) at 0.2 mM and the corresponding 0.1 mM probe. Commercial mixtures of 20×Taqman Gene Expression Assay (ThermoFisher) primers corresponding to the mRNAs to be quantified bearing the FAM reporter are used (Table 7). The ribophosphoprotein acid gene RPLP0 coding for a ribosomal protein, invariant under the different conditions, was chosen as the normalizing gene using the VIC reporter. The primers and Taqman probe used for amplification of RPLP0 are as follows: m181PO.F (5′-CTCCAAGCAGATGCAGCAGA-3′ (SEQ ID NO: 7)), m267PO.R (5′-ACCATGATGATGCG CAAGGCCAT-3′ (SEQ ID NO: 8)) and m225PO.P (5′-CCGTGGTGCTGATGGGGGGCAAGA A-3′ (SEQ ID NO: 9)). DNA samples are either cDNA samples obtained after reverse transcription or viral DNA. The PCR reaction takes place in 384-well plates, each well is duplicated in the quantities shown in the Table 3.

TABLE 3
Reaction mixture for quantitative PCR

| Product | Quantity |
|---|---|
| DNA | 50 ng |
| Thermo Scientific | 1X |
| Absolute qPCR ROX Mix | |
| TaqMan Gene Expression 20X FAM | 0.5X |
| Standardizer RPLPO 20X VIC | 0.5X |
| Water | qsp 10 μL |

The following PCR program is applied: pre-incubation 15 minutes at 95° C., then 45 amplification cycles of 15 seconds at 95° C. followed by 1 minute at 60° C. using the LightCycler480 (Roche).

TABLE 4
List of Taqman Gene Expression primers used

| Gene | Reference | Gene | Reference |
|---|---|---|---|
| miR 142-3p | hsa-miR-142-3p | Tgfb1 | Mm01178820_m1 |
| miR 21 | hsa-miR-21 | Ctnnb1 | Mm004893039_m1 |
| miR 31 | mmu-miR-31 | mCilp | Mm00557687_m1 |
| Col1a1 | Mm00801666_g1 | hCilp | Hs01548460_m1 |
| Myh8 | Mm01329494_m1 | GFP | Mr 03989638 |
| Tmem8c | Mm00481256_m1 | mLtbp2 | Mm01307379_m1 |
| Nppa | Mm01255747_g1 | hLtbp2 | Hs00166367_m1 |
| Myh7 | Mm0060555_m1 | mWisp2 | Mm00497471_m1 |
| Myh6 | Mm00440359_m1 | hWisp2 | Hs1031984_m1 |
| Fn | Mm01256744_m1 | mDkk3 | Mm00443800_m1 |
| Vim | Mm01333430_m1 | hDkk3 | Hs00247429_m1 |
| Col1a1 | Mm00801666_g1 | mSfrp2 | Mm01213947_m1 |
| Col3a1 | Mm00802300_m1 | hSfrp2 | Hs00293258_m1 |
| Timp1 | Mm01341361_m1 | | |

The cycle quantification is calculated with the LightCycler® 480 SW 1.5.1 software (Roche) using the maximum second derivative method. Quantitative PCR results are expressed in terms of “Cq”, the number of cycles after which a threshold fluorescence value is reached. This value is then normalized to the value obtained for the reference gene RPLP0.

Mitochondrial PCR kits (PAMM-087Z) and WNT (PAMM-243Z) and TGF-B (PAMM-235Z) target screening are used according to the manufacturer's instructions (RT2 Profiler PCR Arrays, Qiagen). RNA extraction is performed from frozen tissue using the RNasy® Micraoarray tissue kit (Qiagen) and processed with the RNase-Free DNase set (Qiagen). The cDNA is obtained from 500 ng RNA using the RT2 first strand kit (Qiagen) and is used as a template for PCR. The qRT-PCR is performed using the LightCycler480 (Roche, Basel, Switzerland).

1.10 RNA Sequencing

The samples used for sequencing are total RNA extracted with TRIzol, treated twice with DNAse and having an INR quality >7. 7. Samples of 2 μg RNA at 100 ng/μL were sent for sequencing to Karolinka Institutet. The sequencing library used was prepared with the TruSeq Stranded Total RNA Library Prep Kit (Illumina) and sequencing was performed according to the Illumina protocol. The reads are associated using Fastq-pair and aligned to the mouse genome (mm10) using STAR align. The number of reads is proportional to the abundance of corresponding RNAs in the sample. The sequencing platform then provides several files per sample, containing the alignment files in barn format, the list of genes identified with the number of reads for each sample compared and the list of genes accompanied by a normalized numerical count value expressed in fragments per kb per million reads (FPKM).

Once the files containing the lists of sequenced transcripts were received, the first step in comparing the samples with each other was to merge the files of the different samples. The goal is to obtain a single table containing, for each transcript identified in the study, its number of reads in each sample. Then, an analysis under the R software was performed with the DESeq2 package: from the number of reads, the samples are normalized, and the differential gene expression for each sample is calculated with respect to its control. The expression difference values (or fold change) are expressed in binary logarithm (log2.FC), they are associated with their adjusted Pvalue padj. Then, a sorting step was performed to remove: genes containing less than 10 reads under all conditions, genes with no significant padj, genes with a log2.FC between −0.5 and 0.5 for all conditions. The final table was used to identify genes expressed significantly differentially between the different conditions.

The alignment of the reads on the mouse genome (mm10) can be observed by viewing the barn files with the Integrative Genomic Viewer (IGV) software. Different R packages are used for the graphical representation of RNAseq results. For Venn diagrams, the Venn Diagram package is used. For Volcano Plots, the ggplot2 package is used. The Ingenuity Pathway Analysis software (IPA, Qiagen) and the gene ontology classification system PANTHER are used to visualize the deregulated signaling pathways in the dataset.

1.11 Cardiac Function Analysis: Ultrasound

The mice are anaesthetized by inhalation of isoflurane and placed on a heating platform (VisualSonics). Temperature and heart rate are continuously monitored. The image is taken by a Vevo 770 high-frequency echocardiograph (VisualSonics) with 707B probe. Ultrasound measurements in 2D mode and M mode (motion) are taken along the large and small parasternal axis at the widest level of the left ventricle. Quantitative and qualitative measurements are performed using the Vevo 770 software. The mass of the left ventricle is estimated using the following formula:

Mass of the left ventricle(g)=0.85(1.04(((diameter of the left ventricle at the end of diastole+thickness of the intraventricular septum at the end of diastole+thickness of the posterior wall at the end of diastole)³−diameter of the ventricle at the end of diastole3)))+0.6

For each ultrasound of a mouse heart, about 5 measurement points are taken. The measuring point corresponding to the maximum size of the left ventricle in diastole is then used, as it represents the maximum dilatation that the mouse heart can reach.

1.12 Viral Vectors

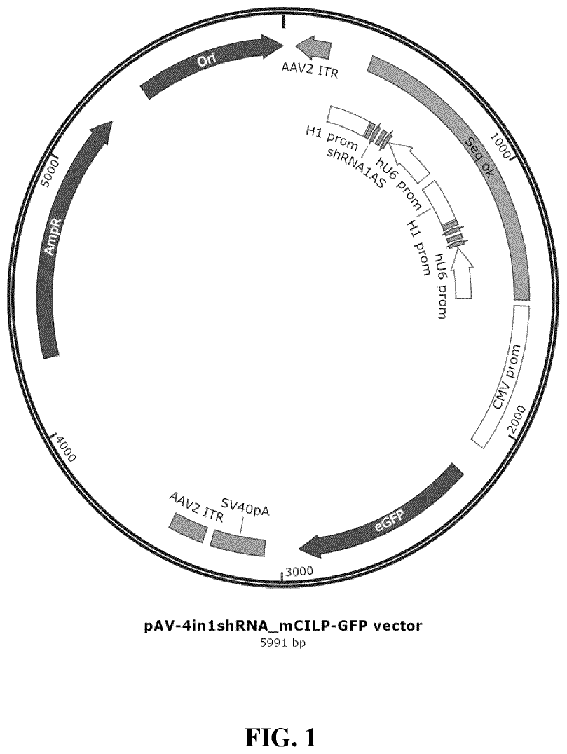

The shRNA plasmid constructs for transgene inhibition have been ordered from Vigene Bioscience. They are constructs comprising 4 individual shRNA sequences and the GFP reporter gene. The sequences selected for each gene are described in the Table 5. The plasmids are constructed according to the model in FIG. 1.

TABLE 5
ShRNA sequences

| Sh-CILP-1 targeted sequence | SEQ ID NO |
|---|---|
| GCATGTGCCAGGACTTCATGC | 1 |
| GGTTCCGAGTTCCTGGCTTGT | 2 |
| GCCTGAAGTCAGCTACCATCA | 3 |
| GCTGGATCCCTCCCTATAA | 4 |

1.13 Production of Plasmids

Plasmids are produced by transforming 45 μL of DH10B bacteria with 2 μL of plasmid. Thermal shock is achieved by alternating 5 minutes in ice, 30 seconds at 42° C. and cooling on ice. Then, 250 μL of SOC (super optimal broth) medium is added before incubation at 37° C. for 1 h under agitation. The bacteria thus transformed are isolated by a 50 μL culture over night at 37° C. on a box of LB (lysogeny broth) containing ampicillin in order to select the bacteria having integrated the plasmid. A clone is transplanted the next day for a preculture of a few hours at 37° C. in 3 mL of LB medium containing antibiotic. Samples are kept for freezing in 50% glycerol. An overnight culture is then performed in 2 L Erlenmeyer containing 500 mL of antibiotic-containing medium and 1 mL of the preculture at 37° C. A NucleoBond PC 2000 EF (Macherey Nagel) kit is then used according to the supplier's instructions to purify the plasmids which are then sterilized by filtration at 0.22 μm and assayed with Nanodrop.

An enzymatic digestion is performed to check the plasmid with the restriction enzymes SMA1 and NHE1. A mixture containing 1 μg of DNA, 2 μL of buffer fast digest green 10×, 1 μl of each enzyme in sterile water for a total amount of 20 μl is stirred for 20 min at 37° C. A 1% agarose gel in TAE (Tris, Acetate, EDTA) containing SYBR™ Safe DNA Gel Stain (Invitrogen) is poured before depositing the digest products and the size marker O'GeneRuler™ DNA Ladder mix.

1.14 Vector Production

The tri-transfection method is used to prepare recombinant viruses. HEK293 cells are used as packaging cells to produce the virus particles. Three plasmids are required: the vector plasmid, which provides the gene of interest, the helper plasmid pAAV2-9_Genethon_Kana (Rep2Cap9), which provides the Rep and Cap viral genes, and plasmid pXX6, which contains adenoviral genes and replaces the co-infection by an adenovirus, necessary for AAV replication. The cells are then lysed and the viral particles are purified. Vectors are produced in suspension.

Cell inoculation (day 1): Use of HEK293T clone 17 cells at confluence, inoculated in 1 L agitation flasks: 2E5 cells/mL in 400 mL of F17 medium (Thermo Fisher scientific). Incubation under agitation (100 rpm) at 37° C.-5% CO2-humid atmosphere.

Cell Transfection (Day 3): Cells are counted and cell viability is measured on Vi-CELL after 72 h of culture. The transfection mix is prepared in Hepes buffer at 10 mg/mL for each plasmid according to its concentration, size and the amount of cells in the flask, the ratio of each plasmid is 1. Incubation 30 minutes at RT after the addition of transfection agent and homogenization of the solution. The transfection mixture and 3979 μL of culture medium (F17 GNT Modified) are transferred to shaker flasks containing 400 mL of culture which are incubated under agitation (130 rpm) at 37° C.-5% CO2-wet atmosphere. After 48 h, treatment of the cells with benzonase: dilution of Benzonase (25 U/mL final) and MgCl2 (2 mM final) in F17 medium, addition of 4 mL per flask.

Viral vector harvest (day 6): Cells are counted and cell viability is measured on Vi-CELL, then 2 mL of triton X-100 (Sigma, 1/200th dilution) are added before incubating 2.5 hours at 37° C. with agitation. The erlenmeyers are transferred to Corning 500 mL and centrifuged at 2000 g for 15 minutes at 4° C. Supernatants are transferred to new Corning 500 mL before adding 100 mL of PEG 40%+NaCl and incubating 4 h at 4° C. The suspension is centrifuged at 3500 g for 30 minutes at 4° C. The pellets are resuspended in 20 mL TMS at pH 8 (Tris HCl at 50 mM, NaCl at 150 mM and MgCl2 at 2 mM, diluted in water) and transferred to Eppendorf 50 mL before the addition of 8 μL benzonase. After 30 min incubation at 37° C., the tubes are centrifuged at 10,000 g for 15 min at 4° C.

Cesium Chloride Gradient Purification: To achieve the gradient, 10 mL of cesium chloride at a density of 1.3 grams/mL is deposited in ultracentrifuge tubes. A volume of 5 mL of cesium chloride at a density of 1.5 grams/mL is then placed underneath. The supernatant is gently deposited on top of the cesium chloride and the tubes are ultracentrifuged at 28,000 RPM for 24 hours at 20° C. Two bands are observed: the upper band contains the empty capsids and the lower band corresponds to the full capsids. Both strips are collected avoiding the removal of impurities. The sample is mixed with cesium chloride at a density of 1.379 g/mL in a new ultracentrifuge tube and then ultracentrifuged at 38,000 RPM for 72 hours at 20° C. The solid capsid strip is removed.

Concentration and filtration: The removal of cesium chloride from the viral preparation and the concentration are carried out on Amicon®(Merck) filters. On Amicon®(Merck) filters, the vectors are concentrated by ultrafiltration with a cut-off of 100 kDa. Amicon membranes are first hydrated with 14 mL 20% ethanol, centrifuged 2 min at 3000 g, then equilibrated with 14 mL PBS, centrifuged 2 min at 3000 g, and then with 14 mL 1,379 ClCs. The collected solid capsid strip is placed on the filters and centrifuged 4 min at 3000 g. 15 mL PBS 1×+F68 formulation buffer is added, before further filtration 2 min at 1500 g. The three previous steps are repeated 6 more times before recovering the last concentrate. The samples are then filtered at 0.22 μm.

Titration: The vector is then assayed by quantitative PCR.

1.15 Statistics

In all statistical analyses, the differences are considered significant at P<0.05 (*), moderately significant at P<0.01 (**) and highly significant at P<0.001 (***), with P=probability. Bar graphs are shown as means+SEM standard deviations. The graphs are made using the GraphPad software.

Analysis of the distribution of fibrosis over the whole heart: In order to ensure that the fibrosis is homogeneous in the heart (H0 hypothesis), the inventors randomly drew 20 values from the 483 fibrosis ratio values. These values were compared 10 to 10 with a Wilcoxon test (Software R) to obtain a p-value. This operation was repeated 1000 times, resulting in 1000 p-values. Among these values some are below 0.05 showing that in some cases our hypothesis of fibrosis invariance is not valid. Out of the 1000 statistical tests, the inventors counted how many gave a value below 0.05. The inventors repeated the entire process 100 times to obtain an average of the percentage for which our H0 hypothesis is false. This average is 4%. This means that our hypothesis is valid 96% of the time, and therefore corresponds to an overall p-value of 0.04, which is statistically acceptable.

2. Results

The inventors wanted to determine whether there were common gene expression modifications between two cardiomyopathy models: the DeltaMex5 model and the DBA/2-mdx model as well as the age at which these deregulations are established and their specificity. To do so, the inventors conducted a comparative study of transcriptome at different ages.

2.1 RNAseq Analysis of the Two Models of Cardiomyopathy

Total RNAseq (RNAseq) sequencing analysis was performed on heart samples from DeltaMex5 and DBA/2-mdx mice and their controls at early and late age of cardiac involvement. For DeltaMex5 mice, ages of 1 and 4 months were chosen, and for DBA/2-mdx mice, ages of 1 and 6 months. The main aim here was to identify genes present when the pathology is established that would be common to both cardiomyopathy models.

The sequencing was done according to the Illumina protocol. The differential expression of genes for each sample is calculated in relation to its control from their read number (>10). The expression difference values (or fold change) are expressed in binary logarithm (log2.FC) and are associated with their adjusted Pvalue padj. Genes expressed significantly differentially between different conditions are determined by a log2.FC>|0.5| and a padj<0.05.

The volcano plot of the RNAseq data allows visualization for each condition of the distribution of genes and the extent of gene deregulation in the heart, as well as the extent of gene expression. The list of the 30 most deregulated genes at 4 months is presented in the Table 6.

TABLE 6
Top 30 most deregulated genes in the core
of the DeltaMex5 model at 4 months.

| | | | Average | Average |
|---|---|---|---|---|
| Gene | log2FC | padj | DeltaMex5 | C57BL/6 |
| Spp1 | 6.60 | 5.28E−128 | 2162.74 | 6.65 |
| Gm42793 | 4.82 | 3.66E−46 | 212.55 | 0.00 |
| Cilp | 4.77 | 4.70E−278 | 3357.31 | 109.77 |
| Ltbp2 | 4.74 | 2.97E−174 | 2206.18 | 68.03 |
| Gpnmb | 4.68 | 1.57E−97 | 764.57 | 19.98 |
| Sprr1a | 4.33 | 1.41E−36 | 222.75 | 1.39 |
| Tnc | 4.28 | 2.99E−33 | 4363.96 | 38.66 |
| Gm6166 | 4.24 | 5.99E−39 | 171.45 | 2.01 |
| 8030451A03Rik | 4.22 | 4.64E−35 | 206.04 | 1.72 |
| D030025P21Rik | 4.02 | 5.44E−36 | 153.86 | 2.87 |
| Timp1 | 3.98 | 5.70E−52 | 625.54 | 24.25 |
| Col12a1 | 3.82 | 2.14E−60 | 989.43 | 50.08 |
| Col8a2 | 3.74 | 3.28E−40 | 247.08 | 10.59 |
| Sfrp2 | 3.62 | 2.08E−55 | 501.17 | 30.20 |
| Thbs4 | 3.61 | 3.79E−121 | 919.77 | 66.85 |
| Ptn | 3.40 | 1.25E−35 | 290.91 | 17.60 |
| Postn | 3.35 | 1.65E−21 | 12040.11 | 414.51 |
| Mfap4 | 3.31 | 3.99E−57 | 428.05 | 35.05 |
| Piezo2 | 3.27 | 2.87E−31 | 209.01 | 13.45 |
| Gm26771 | 3.26 | 9.27E−27 | 132.29 | 7.36 |
| Col3a1 | 3.22 | 1.72E−61 | 40685.43 | 3682.59 |
| Col14a1 | 3.20 | 9.47E−116 | 2081.22 | 207.54 |
| Ctss | 3.19 | 3.04E−59 | 1773.00 | 162.81 |
| Trem2 | 3.16 | 2.89E−28 | 259.21 | 17.93 |
| Atp6v0d2 | 3.15 | 2.67E−17 | 58.07 | 0.67 |
| Apol7d | 3.15 | 5.17E−32 | 541.35 | 41.67 |
| AC125167.1 | 3.12 | 3.11E−46 | 1533.67 | 141.84 |
| Lgals3 | 3.10 | 6.97E−19 | 747.38 | 35.14 |
| Mpeg1 | 3.09 | 8.80E−23 | 2205.83 | 143.04 |
| Dkk3 | 3.01 | 2.78E−32 | 299.63 | 27.12 |

Underlined = model specific.

One of the most increased gene in the heart of the DeltaMex5 model at 4 months is the Cilp gene, coding for Cartilage Intermediate Layer Protein (log2FC=4.77, P=4.70E-278), a negative regulator of the TGF-β pathway Shindo, K. et al. 2017. International Journal of Gerontology 11, 67-74). At 1 month, the number of deregulated genes is much smaller and the deregulated genes are deregulated to a lesser extent with a maximum log2FC of 1.

For the DBA/2-mdx model, the list of the 30 most deregulated genes is presented in the Table 7.

TABLE 7
Top 30 most deregulated genes in the core
of the DBA/2-mdx model at 6 months.

| | | | Average | Average |
|---|---|---|---|---|
| Gene | log2FC | padj | DBA/2-mdx | DBA/2 |
| Ighg2c | 3.99 | 2.34E−40 | 127.71 | 0.00 |
| Tnc | 3.76 | 1.89E−111 | 1637.90 | 96.86 |
| Cilp | 3.27 | 4.59E−70 | 760.53 | 62.18 |
| Sprr1a | 3.02 | 7.35E−22 | 75.33 | 1.37 |
| Mt2 | 2.98 | 3.69E−44 | 425.98 | 39.53 |
| Timp1 | 2.83 | 1.53E−19 | 735.88 | 34.09 |
| 8030451A03Rik | 2.65 | 5.15E−18 | 77.45 | 4.44 |
| Serpina3n | 2.60 | 9.78E−18 | 2728.99 | 200.98 |
| Chile1 | 2.54 | 1.01E−18 | 173.35 | 15.73 |
| Hamp2 | −2.51 | 2.58E−38 | 61.43 | 427.96 |
| Lrp8 | 2.47 | 1.81E−16 | 132.79 | 11.37 |
| Saa3 | 2.41 | 6.08E−13 | 46.74 | 0.65 |
| Fam46b | 2.36 | 5.00E−34 | 511.91 | 83.18 |
| Per2 | 2.35 | 8.14E−32 | 292.28 | 47.22 |
| Fgl2 | 2.34 | 4.46E−65 | 3216.00 | 585.39 |
| Lox | 2.32 | 7.38E−52 | 919.81 | 166.22 |
| Crlf1 | 2.30 | 1.98E−19 | 172.27 | 24.17 |
| Postn | 2.28 | 6.18E−21 | 9086.68 | 1378.81 |
| Ereg | 2.27 | 3.18E−12 | 58.87 | 3.76 |
| Cfb | 2.27 | 4.86E−41 | 632.26 | 115.30 |
| Nxpe5 | 2.27 | 4.06E−28 | 215.52 | 36.33 |
| Gm20547 | 2.27 | 6.53E−48 | 712.93 | 133.02 |
| Ccl6 | 2.26 | 1.05E−64 | 1020.70 | 197.87 |
| Ccl9 | 2.24 | 4.79E−43 | 492.52 | 93.30 |
| Pak3 | 2.20 | 3.81E−15 | 117.72 | 15.86 |
| Mmp3 | 2.17 | 7.28E−35 | 967.23 | 187.88 |
| Srpx | 2.17 | 9.38E−31 | 366.18 | 70.08 |
| Clec4d | 2.16 | 1.12E−12 | 66.61 | 7.53 |
| Ccl7 | 2.16 | 2.68E−18 | 140.95 | 22.98 |
| He33 | 2.15 | 2.60E−32 | 353.56 | 69.27 |

Underlined = model specific.

In the DBA/2-mdx model at 6 months, the inventors find in the first 5 positions Culp gene as one of the most deregulated gene. At 1 month, the number of deregulated genes is already high and the most deregulated genes exceed a log 2FC of 3.

The Venn diagram representation of RNAseq results allows the visualization of the numbers of common or specific deregulated genes in a model or a stage of disease progression. Of the 46,717 genes included in the RNAseq analysis, 4,850 genes were found to be significantly deregulated (llog2FCI>0.5 and pvalue<0.05) in either model at early or late age of cardiac involvement compared to control. At an early age, the heart of DeltaMex5 mice has only 44 deregulated genes, whereas the heart of DBA/2-mdx mice already has 2,186, with only 4 genes in common in both models. At a later age, the DeltaMex5 heart has 2,621 deregulated genes and the DBA/2-mdx heart has 2,202, of which 1,175 are common to both models, of which 708 genes are specific for the advanced age of cardiomyopathy. Only 9 genes are specific for the DeltaMex5 model, while 232 are specific for the DBA/2-mdx model. Of all the deregulated genes, a greater proportion of the genes are over-expressed rather than under-expressed.

The majority of the most over-expressed genes are common between the two models. However, genes deregulated in the hearts of DeltaMex5 mice at 4 months are more strongly deregulated than genes deregulated in the hearts of DBA/2-mdx mice at 6 months (log 2FC maximum of 4 versus 6.6). It was also observed that, although the cardiac involvement between the two models was different, the transcriptional deregulations associated with them mostly involved the same genes and signalling pathways at a late stage.

To complete this analysis, the Ingenuity Pathway Analysis (IPA, Qiagen) software, which uses a repository of biological interactions and functional annotations to help interpret the data into biological mechanisms was used. At one month of age, no increase in signaling pathways was identified in the hearts of DeltaMex5 and DBA/2-mdx mice. Analysis by IPA allowed to highlight the biological functions whose genes are most represented in the deregulated genes in an advanced phase. In first position in both models, more than 150 genes involved in cardiovascular disease were found in the RNAseq analysis. In second position, more than 150 deregulated genes are categorized in the family of lesions and abnormalities on an organ. Finally, in third position, nearly 200 genes related to the function and development of the cardiovascular system were found.

The inventors also used another function of the IPA software to determine the toxicity associated with the observed changes in gene expression, and this only in the advanced phases. Many deregulated genes were identified: 86 genes associated with cardiac enlargement in the DeltaMex5 model and 85 in the DBA/2-mdx model, 45/48 genes that could lead to cardiac dysfunction, 38/36 genes in cardiac dilatation, 27/28 genes in cardiac fibrosis and 35/37 in cardiac necrosis.

The PANTHER gene ontology classification system was also used to determine the most deregulated signalling pathways in the late-stage models. In both models, the perturbations appear to be very similar as seen in the analysis of the Venn Diagrams. The first two pathways found are similar in both models and include the chemokine and cytokine mediated inflammation signalling pathway with nearly 70 genes involved, and the integrin pathway with more than 50 genes involved. The inflammation is likely the result of cellular damage associated with cardiomyopathy. Integrins play a major role in the transmission of mechanical forces between membranes and the adaptation to these forces in cardiomyocytes. Interestingly, the TGF-β pathway is found in 22nd and 19th position, with more than 15 deregulated genes, to which Clip, one of the most over-expressed genes, belong. Among the other canonical pathways that are not represented in the graph, the most decreased pathways in the models are oxidative phosphorylation and mitochondrial function, with a decrease of factors in each of the 5 mitochondrial complexes of the respiratory chain. There is also the peroxisome proliferator-activated receptor pathway (PPARγ), which has a role in cardiac metabolism.

2.2 Validation of Deregulated Genes

The deregulation of CILP-1, one of the most deregulated genes was evaluated under different conditions. CILP-1 is not overexpressed in the DeltaMex5 model at 1 month but is over-expressed in the later age of the disease (Table 8).

TABLE 8
Deregulation of CILP gene in the models.

| | | Cilp |
|---|---|---|
| DeltaMex5 | log2FC | 0.16 |
| 1 month | padj | 0.00E+00 |
| | Average DeltaMex5 | 560 |
| | Average C57BL/6 | 136 |
| DeltaMex5 | log2FC | 4.77 |
| 4 months | Padj | 4.70E−278 |
| | Average DeltaMex5 | 3357 |
| | Average C57BL/6 | 110 |
| DBA/2-mdx | log2FC | 0.63 |
| 1 month | padj | 2.30E−01 |
| | Average DBA/2-mdx | 417 |
| | Average DBA/2 | 111 |
| DBA/2-mdx | log2FC | 3.27 |
| 6 months | padj | 4.60E−70 |
| | Average DBA/2-mdx | 761 |
| | Average DBA/2 | 62 |

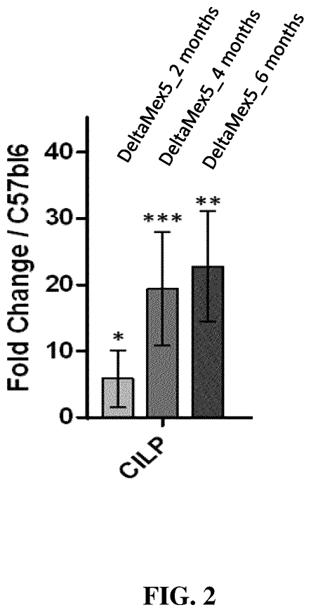

Validation of RNAseq data was then performed on cores of the DeltaMex5 model at different ages (2, 4 and 6 months) by an individual qPCR to confirm their overexpression and assess their modification over time. CILP-1 gene is significantly overexpressed from 2 months in the model, and gene overexpression increases progressively with age (FIG. 2).

2.3 Modulation of CILP-1 Gene Expression

The inventors then wanted to assess the impact of modulation of CILP-1 on the cardiac phenotype of the model. An evaluation of the consequences of in vivo gene transfer of shRNA-CILP-1 on fibrotic status and cardiac function was performed on the DeltaMex5 model. The approaches that have shown an interest in the DeltaMex5 model are currently being applied to the DBA/2-mdx.

2.4 Gene Transfer Approaches

The strategy chosen for inhibiting CILP-1 expression is the use of shRNA. shRNA are small RNAs with a hairpin structure. Their action is based on the principle of interfering RNA, neutralizing the messenger RNA of the target. The inventors have chosen 4-in-1 shRNAs for enhanced efficiency of transgene neutralization wherein four individual shRNA sequences are grouped together in a plasmid. The shRNAs were selected using Thermofisher's RNAi Designer tool. The 4 shRNAs with the best specific recovery score for the gene of interest were selected. They were then ordered from Vigene Bioscience, under the control of H1 and U6 ubiquitous promoters.

1-month-old mice were injected intravenously at a dose of 2e¹¹ vg/mouse (equivalent to a dose of 1e¹³ vg/kg for a mouse of approximately 20 g) or by PBS. After 3 months of vector expression, the hearts of the mice were ultrasonographed prior to collection. The overall, histological and functional consequences on the heart were then studied.

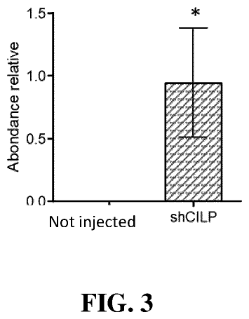

Expression of the vectors AAV9-4in1shRNA-mCILP-GFP is detected using the GFP reporter gene which is present only in mice injected by the vector (FIG. 3). These RT-qPCR assays confirm the presence of the transgenes 3 months after vector injection.

2.4.1 Morphological Evaluation

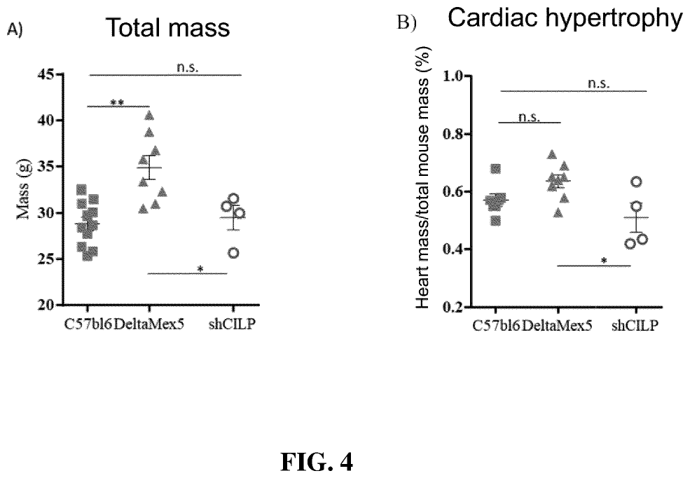

Mouse mass was significantly decreased in mice treated with the AAV9-4in1shRNA-mCILP-GFP vector (29.5±1.31 g, n=4, P=0.027) (FIG. 4A). Heart hypertrophy, as measured by the ratio of heart mass to total mouse mass, in mice treated with the AAV9-4in1shRNA-mCILP-GFP vector was significantly decreased compared to untreated DeltaMex5 mice (0.51±0.05, n=4, P=0.022) and became comparable to C57BL/6 mice (FIG. 4B).

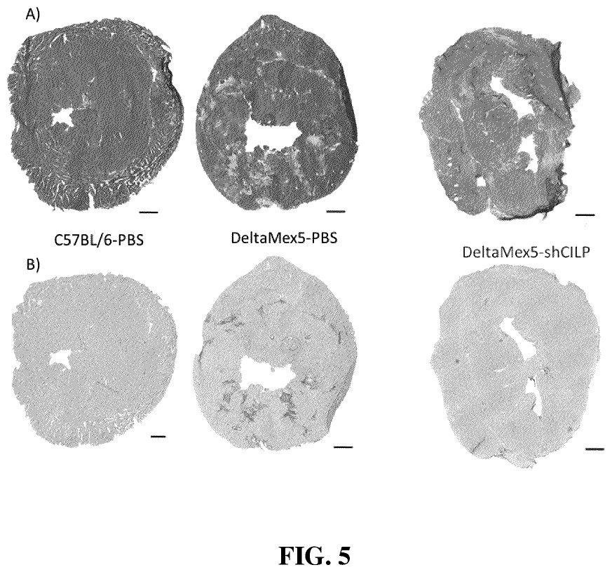

Histological analyses were then performed on the hearts of the mice. HPS staining revealed persistence of the damaged tissue in mice treated with AAV9-4in1shRNA-mCILP-GFP (FIG. 5A). The observation of the slices indicates that the fibrosis in tissue visualized by collagen staining with Sirius Red on AAV-treated samples with shRNA is decreased compared to DeltaMex5 control mice (FIG. 5B).

2.4.2 Functional Evaluation

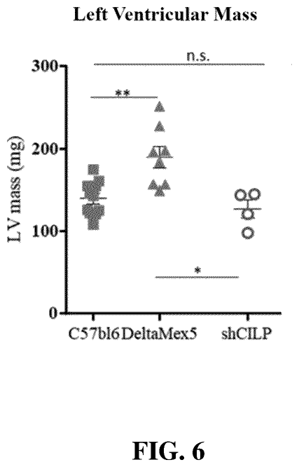

Ultrasound analyses of cardiac function were performed at 4 months, after 3 months of vector expression (FIG. 6). A significant variation in the estimated left ventricular mass in mice injected with AAV9-4in1shRNA-mCILP-GFP is observed with a decrease of almost 40% compared to DeltaMex5 controls (127±11.05 mg, n=4, versus 190±12.77 mg, n=8, P=0.01).

2.4.3 Molecular Evaluation

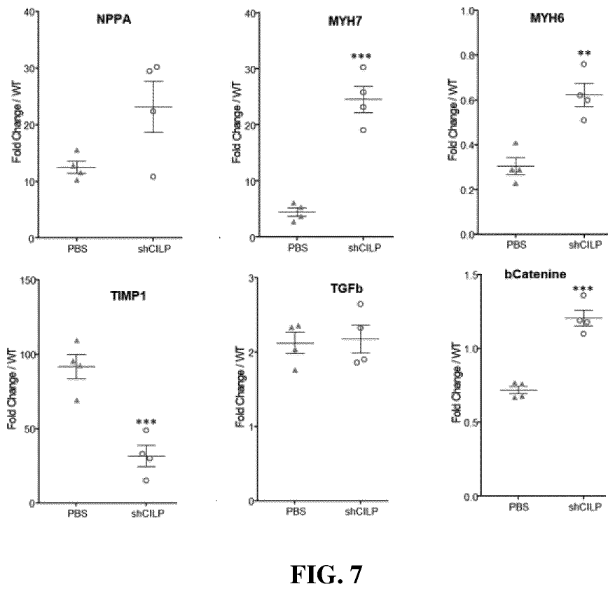

In mice injected with the AAV9-4in1shRNA-mCILP-GFP vector, Myh7 was significantly increased compared to PBS mice (24.59±2.35, P<0.001), Myh6 was also increased (0.62±0.05, P=0.003). The β-catenin which was unchanged between DeltaMex5 and C57BL/6 mice was slightly increased (1.21±0.06, P<0.001). Only Timp1 was significantly decreased in injected mice compared to DeltaMex5-PBS mice (31.67±6.98, P=0.001) (FIG. 7).

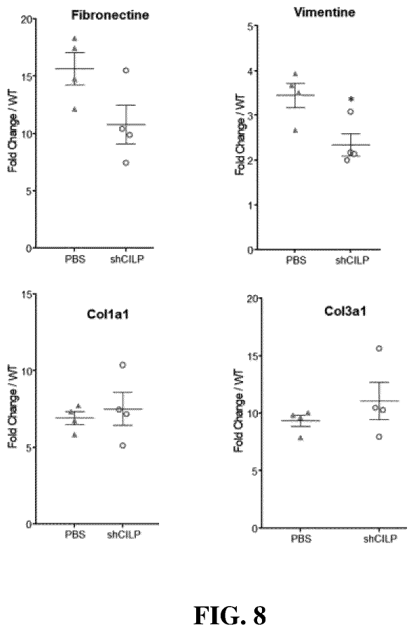

Fibrosis RNA tissue markers (Fibronectin, Vimentin, Collagen 1a1 and Collagen 3a1) were also measured by RT-qPCR. In mice injected with the AAV9-4in1shRNA-mCILP-GFP vector, vimentin, a marker of fibrosis, was significantly decreased (2.35±0.25, P=0.02) (FIG. 8). Vimentin was normalized to C57BL/6 mice.

1-15. (canceled)

16. A method of treating dilated cardiomyopathies in a patient in need thereof, comprising administering to the patient a therapeutically effective amount of a CILP-1 inhibitor.

17. The method according to claim 16, wherein said CILP-1 inhibitor is a nucleic acid interfering with CILP-1 expression.

18. The method according to claim 16, wherein said CILP-1 inhibitor is a shRNA interfering with CILP-1 expression.

19. The method according to claim 16, wherein said CILP-1 inhibitor is a shRNA interfering with CILP-1 expression, which is encoded by a nucleic acid construct.

20. The method according to claim 19, wherein said nucleic acid construct comprises at least one sequence selected from the group consisting of: SEQ ID NO: 1 to 4.

21. The method according to claim 19, wherein said nucleic acid construct comprises the sequences SEQ ID NO: 1 to 4.

22. The method according to claim 19, wherein the nucleic acid construct is packaged into a viral particle.

23. The method according to claim 19, wherein the nucleic acid construct is packaged into an adeno-associated viral (AAV) particle.

24. The method according to claim 19, wherein said nucleic acid construct is packaged into an AAV particle and further comprises 5′-ITR and 3′-ITR of AAV-2 serotype or a 5′ITR and a 3′ITR corresponding to the serotype of the selected AAV particle.

25. The method according to claim 19, wherein said nucleic acid construct is packaged into an AAV particle comprising an AAV capsid protein derived from AAV serotypes selected from the group consisting of: AAV serotypes 1, 6, 8, 9 and AAV9.rh74.

26. The method according to claim 19, wherein said nucleic acid construct is packaged into an AAV particle comprising an AAV capsid protein derived from AAV-9.rh74 serotype.

27. The method according to claim 19, wherein said nucleic acid construct is packaged into a viral particle that is administered intravenously.

28. The method according to claim 16, wherein said dilated cardiomyopathy is a genetically induced cardiomyopathy caused by mutation(s) in a gene selected from the group consisting of: laminin, emerin, fukutin, fukutin-related protein, desmocollin, plakoglobin, ryanodine receptor 2, sarcoplasmic reticulum ca(2+) ATPase 2 isoform alpha, phospholamban, lamin a/c, dystrophin, telethonin, actinin, desmin, sarcoglycans, titin, myosin, RNA-binding motif protein 20, BCL-2 associated athanogene 3, desmoplakin, sodium channels, cardiac actin, cardiac troponin and tafazzin.

29. The method according to claim 16, wherein said dilated cardiomyopathy is a genetically induced dilated cardiomyopathy caused by mutation in titin or dystrophin gene.

30. The method according to claim 16, comprising administering a pharmaceutical composition comprising the CILP-1 inhibitor and a pharmaceutical excipient.


SEQUENCE LISTING (9 sequences)

SEQ ID NO: 1 - DNA, 21 bp, Artificial Sequence
  interfering sequence #1
gcatgtgccaggacttcatgc

SEQ ID NO: 2 - DNA, 21 bp, Artificial Sequence
  Interfering sequence #2
ggttccgagttcctggcttgt

SEQ ID NO: 3 - DNA, 21 bp, Artificial Sequence
  Interfering sequence #3
gcctgaagtcagctaccatca

SEQ ID NO: 4 - DNA, 21 bp, Artificial Sequence
  Interfering sequence #4
gctggatccctccctctataa

SEQ ID NO: 5 - protein, 16 aa, Artificial sequence
  Titanium IS7-1
LVEEPPPREVVLKTSC

SEQ ID NO: 6 - protein, 15 aa, Artificial sequence
  M10-1 Titanium
IEALPSDISIDEGKV

SEQ ID NO: 7 - DNA, 20 bp, Artificial sequence
  m181PO.F
ctccaagcagatgcagcaga

SEQ ID NO: 8 - DNA, 23 bp, Artificial sequence
  m267PO.R
accatgatgatgcgcaaggccat

SEQ ID NO: 9 - DNA, 25 bp, Artificial sequence
  m225PO.P
ccgtggtgctgatggggggcaagaa